(19) United States
(12) Patent Application Publication
(10) Pub. No.: US 2010/0092468 A1
(43) Pub. Date: Apr. 15, 2010

(54) METHOD FOR PREDICTING THERAPEUTIC RESPONSIVENESS TO TNF-ALPHA BLOCKING AGENTS

(76) Inventors: Corinne Miceli, Chevilly La Rue (FR); Xavier Mariette, Rueil Malmaison (FR)

Correspondence Address: STITES & HARBISON PLLC, 1199 NORTH FAIRFAX STREET, SUITE 900, ALEXANDRIA, VA, 22314, US

(21) Appl. No.: 12/513,738

(22) PCT Filed: Nov. 11, 2006
(86) PCT No.: PCT/IB2006/003165

§ 371 Date: Dec. 1, 2009

(51) Int. Cl. C12Q 1/68; A61K 38/17; A61K 39/395; A61P 19/02

(52) U.S. Cl. 424/133.1; 435/6; 514/12; 424/142.1

(57) ABSTRACT

The present invention relates to a method for predicting the responsiveness of a patient to a treatment with a TNF-alpha blocking agent, said method comprising determining the presence or absence of a guanine at position −238, a guanine at position −308, and a cytosine at position −857 of the TNF-alpha gene of said patient, wherein the simultaneous presence of a guanine at position −238, a guanine at position −308, and a cytosine at position −857 of the TNF-alpha gene in both copies of said TNF-alpha gene of said patient is indicative of a lessened likelihood of responsiveness of said patient to a treatment with a TNF-alpha blocking agent with respect to standard responsiveness.

### FIELD OF THE INVENTION

The present invention relates to a method for predicting the response to a treatment with a TNF-alpha blocking agent.

### BACKGROUND OF THE INVENTION

Rheumatoid arthritis (RA) is a chronic, auto-immune and inflammatory polyarthritis which induces joint damage and disability. Studies have led to the recognition of TNF-alpha as one of the cornerstone cytokines involved in synovial inflammatory process. Such results have provided the basis for the development of TNF blocking agents (TBAs) for the treatment of RA. Three TBAs are currently used for RA treatment, one corresponding to a recombinant soluble form of TNF receptor, TNFRSF1B (etanercept), two others corresponding to an anti-TNF-alpha monoclonal antibody: infliximab and adalimumab (ADA). These TBAs act by inhibiting the binding of TNFs to TNF receptors on cell surface and therefore interfering with TNF driven signal transduction pathways. Etanercept binds to both TNF-alpha and TNF-beta (also known as LymphoToxine A, LTA) while infliximab and adalimumab bind to TNF-alpha only.

Various clinical trials with a TBA/methotrexate combination have shown efficacy in 55-75% of RA patients. TBAs reduce joint inflammation, slow down joint damage and improve physical function. Still, 25-45% of the RA patients given a TNF-alpha blocking agent/methotrexate combination do not respond to this treatment. Moreover, TBAs may have side effects and are costly and the efficacy of any given TBA in a given patient is unpredictable.

Taking into account the cost of these treatments, the persisting doubts about potential long term adverse events (infections and cancers) and the availability of other efficient biotherapies in the treatment of RA, identification of predictive factors of response is a key issue.

Because genetic polymorphisms such as HLA-DR haplotypes have been associated with a variable natural course of RA and a heterogeneous response to conventional disease-modifying anti-rheumatic drugs (DMARD), a few studies have attempted to identify genetic markers for TBA efficacy and they have focused on the promoters of several cytokine genes (Kang C P et al. 2006; Mugnier et al. 2003; Cuchacovich et al. 2004, Padyulov et al. 2003). For example, sequence variation in the TNF-alpha gene promoter has been associated with a variable response to infliximab (Mugnier et al. 2003). However, those studies have led to contradictory results, especially those concerning the role of TNF −308A/G polymorphism and/or the shared epitope (Padyulov et al. 2003; Criswell et al. 2004, Mugnier et al. 2003, Cuchacovich et al. 2004, Martinez et al. 2004, Mugnier et al. 2004, Fronseca et al. 2005, Kang et al. 2005, Lee et al. 2006).

### SUMMARY OF THE INVENTION

The inventors have now identified that the haplotype consisting of −238G, −308G and −857C in the TNF-alpha gene may be useful to predict the response to treatment with a TNF-alpha blocking agent.

They have indeed genotyped HLA-DRB1 and three single nucleotide polymorphisms (SNPs) of the TNF-alpha gene, namely −857C/T, −308A/G, and −238A/G, in a large cohort of Caucasian patients with rheumatoid arthritis and treated with adalimumab (ADA), with or without methotrexate (MTX). For each gene, genotypes and haplotypes were tested for association with ACR50 response (50% improvement in symptoms according to the American College of Rheumatology criteria) at week 12 of the treatment. The inventors have demonstrated that none of the three TN F-alpha polymorphisms tested separately in genotypic distribution was associated with the ACR50 response in univariate or multivariate analysis. Conversely, haplotype reconstruction of TNF-alpha locus revealed that the haplotype consisting of −238G, −308G, and −857C (“GGC” haplotype), in an homozygous form (present in almost 50% of patients), was significantly associated with a poorer response to ADA.

Thus, the present invention relates to a method, in particular an in vitro method, for predicting the responsiveness of a patient to a treatment with a TNF-alpha blocking agent, said method comprising determining the presence or absence of a guanine at position −238, a guanine at position −308, and a cytosine at position −857 of the TNF-alpha gene of said patient, wherein the simultaneous presence of a guanine at position −238, a guanine at position −308, and a cytosine at position −857 of the TNF-alpha gene in both copies of said TNF-alpha gene of said patient is indicative of a lessened likelihood of responsiveness of said patient to a treatment with a TNF-alpha blocking agent with respect to standard responsiveness.

The present invention also relates to the use of a TNF-alpha blocking agent for the manufacture of a medicament intended for treating a patient with a TNF-alpha-related disease, wherein said patient does not simultaneously carry a guanine at position −238, a guanine at position −308, and a cytosine at position −857 of the TNF-alpha gene in both copies of said TNF-alpha gene.

The present invention also relates to a method for treating a TNF-alpha-related disease in a patient likely to respond to treatment with a TNF-alpha blocking agent, which method comprises the steps of:
a) determining the presence or absence of a guanine at position −238, a guanine at position −308, and a cytosine at position −857 of the TNF-alpha gene of said patient;
b) administering a therapeutically effective amount of at least one TNF-alpha blocking agent to the patient, if said patient is likely to respond to treatment with a TNF-alpha blocking agent, i.e. if said patient does not simultaneously carry a guanine at position −238, a guanine at position −308, and a cytosine at position −857 of the TNF-alpha gene in both copies of said TNF-alpha gene of said patient.

### DETAILED DESCRIPTION OF THE INVENTION

### Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

As used herein, the term “oligonucleotide” refers to a nucleic acid, generally of at least 10, preferably at least 15, and more preferably at least 20 nucleotides, preferably no more than 100 nucleotides, still preferably no more than 70 nucleotides, and which is hybridizable to a genomic DNA, cDNA, or mRNA. Oligonucleotides can be labelled according to any technique known in the art, such as with radiolabels, fluorescent labels, enzymatic labels, sequence tags, etc. A labelled oligonucleotide may be used as a probe to detect the presence of allelic variants of TNF nucleic acid. Alternatively, oligonucleotides (one or both of which may be labelled) can be used for amplifying a region of a TNF nucleic acid, for instance by PCR (Saiki et al., 1988), to detect the presence of an allelic variant. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989).

The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15 M NaCl, 0.015 M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA: RNA, DNA: RNA, DNA: DNA. For hybrids of greater than 100 nucleotides in length, equations for calculating Tm have been derived (see Sambrook et al., 1989, 9.50-9.51). For hybridization with shorter nucleic acids, i.e., oligonucleotides, the position of mismatches becomes more important, and the length of the oligonucleotide determines its specificity (see Sambrook et al., 1989 II.7-11.8). A minimum length for a hybridizable nucleic acid is at least about 10 nucleotides, preferably at least about 15 nucleotides, and more preferably the length is at least about 20 nucleotides.

In a specific embodiment, the term “standard hybridization conditions” refers to a Tm of 55° C., and utilizes conditions as set forth above. In a preferred embodiment, the Tm is 60° C. In a more preferred embodiment, the Tm is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, an amplification primer is an oligonucleotide for amplification of a target sequence by extension of the oligonucleotide after hybridization to the target sequence or by ligation of multiple oligonucleotides which are adjacent when hybridized to the target sequence. At least a portion of the amplification primer hybridizes to the target. This portion is referred to as the target binding sequence and it determines the target-specificity of the primer. In addition to the target binding sequence, certain amplification methods require specialized non-target binding sequences in the amplification primer. These specialized sequences are necessary for the amplification reaction to proceed and typically serve to append the specialized sequence to the target. For example, the amplification primers used in Strand Displacement Amplification (SDA) include a restriction endonuclease recognition site 5′ to the target binding sequence (U.S. Pat. No. 5,455,166 and U.S. Pat. No. 5,270,184). Nucleic Acid Based Amplification (NASBA), self-sustaining sequence replication (3SR) and transcription based amplification primers require an RNA polymerase promoter linked to the target binding sequence of the primer. Linking such specialized sequences to a target binding sequence for use in a selected amplification reaction is routine in the art. In contrast, amplification methods such as PCR which do not require specialized sequences at the ends of the target, generally employ amplification primers consisting of only target binding sequence.

As used herein, the terms “primer” and “probe” refer to the function of the oligonucleotide. A primer is typically extended by polymerase or ligation following hybridization to the target but a probe typically is not. A hybridized oligonucleotide may function as a probe if it is used to capture or detect a target sequence, and the same oligonucleotide may function as a primer when it is employed as a target binding sequence in an amplification primer. It will therefore be appreciated that any of the target binding sequences disclosed herein for amplification, detection or quantisation of TNF gene may be used either as hybridization probes or as target binding sequences in primers for detection or amplification, optionally linked to a specialized sequence required by the selected amplification reaction or to facilitate detection.

As used herein, the term “TNF-alpha gene” denotes the human gene to which the methods of the invention can apply. The gene is a multifunctional proinflammatory cytokine that belongs to the tumor necrosis factor (TNF) superfamily. Homo sapiens TNF-alpha gene is localized on chromosome 6 at location 6p21.33, the sequence of which is deposited in Genebank under accession number X02910. The TNF promoter sequence is referenced as number 13301 in promoter database located at http://rulai.cshl.edu/cqi-bin/TRED/tred.cgi?process=prominfo&pid=113301. An exemplary genomic sequence of the TNF-alpha gene is shown in SEQ ID NO: 1.

As intended herein the expression “both copies of the TNF-alpha gene” relates to the two alleles of the TNF-alpha gene which are present in the human genome.

The term “TNF-alpha” denotes the tumor necrosis factor—alpha. The human TNF-alpha is a human cytokine encoded by the TNF-alpha gene. This cytokine exists as a 17 kD secreted form and a 26 kD membrane associated form, the biologically active form of which is composed of a trimer of noncovalently bound 17 kD molecules. The structure of human TNF-alpha is described further in, for example, Pennica, D., et al. (1984) Nature 312:724-729; Davis, J. M., et al. (1987) Biochemistry 26:1322-1326; and Jones, E. Y., et al. (1989) Nature 338:225-228. TNF-alpha, a naturally occurring cytokine, plays a central role in the inflammatory response and in immune injury. It is formed by the cleavage of a precursor transmembrane protein, forming soluble molecules which aggregate to form trimolecular complexes. These complexes then bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extravascular tissues.

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. A mutation in the genetic material may also be “silent”, i.e. the mutation does not result in an alteration of the amino acid sequence of the expression product.

The term “Single nucleotide polymorphism” or “SNP” refers to a specific substitution as above defined. The single nucleotide polymorphisms (SNP) consisting of the −857C/T, −308A/G, −238A/G substitutions in the TNF-alpha gene are respectively disclosed by the NCBI accession numbers rs1799724, rs1800629 and rs361525 (http://www.ncbi.nlm.nih.gov/entrez/query.fcgi?CMD=search&DB=snp).

The above SNPs are numbered according to the specific numbering of the TNF-alpha gene, well known to one skilled in the art and notably described in Simmonds et al. (2004) (in particular in FIG. 2 of Simmonds et al.). The origin nucleotide (nucleotide 0) in the above specific numbering corresponds to nucleotide −180 when using standard numbering, wherein nucleotide +1 corresponds to A of the translation initiation codon ATG. According to standard numbering the above SNPs respectively correspond to −1037C/T, −488A/G, and −418A/G. Alternatively, taking SEQ ID NO: 1 as a reference, the SNPs respectively correspond to 33C/T, 582A/G and 652A/G.

The term “haplotype” denotes a set of single nucleotide polymorphisms (SNPs) on a single chromatid that are statically associated. Haplotype may be present in homozygous or heterozygous form.

The term “patient” refers to any subject (preferably human) afflicted with a disease likely to benefit from a treatment with a TNF-alpha blocking agent, in particular a TNF-alpha-related disease.

The term “TNF-alpha-related disease” denotes a disease which is associated with an inflammatory process drove by TNF-alpha. More specifically TNF-alpha-related diseases include diseases and other disorders in which the presence of TNF-alpha in a subject suffering from the disorder has been shown to be or is suspected of being either responsible for the pathophysiology of the disorder or a factor that contributes to a worsening of the disorder.

“TNF-alpha blocking agent” refers to a molecule, such as protein or small molecule that can significantly reduce TNF-alpha properties.

A “responder” or “responsive” patient, or group of patients, to a treatment with a TNF-alpha blocking agent, refers to a patient, or group of patients, who shows or will show a clinically significant relief in the disease when treated with a TNF-alpha blocking agent. The disease activity can be measured according to the standards recognized in the art, such as the “Disease Activity Score” (DAS) or the American College of Rheumatology (ACR) criteria which are measures of the activity of rheumatoid arthritis. The following parameters are included in the calculation:
Number of joints tender to the touch (TEN) based on 28-joint count
Number of swollen joints (SW) based on 28-joint count
Erythrocyte sedimentation rate (ESR)
Patient's assessment of disease activity (VAS; mm) (PATDAI)
   DAS28 provides a continuous variable which does not require reference to a baseline: DAS28=0.56× square root (TEN28)+0.28× square root (SW28)+0.70×(InESR)+0.014×PATDAI. (Prevoo et al. 1995). ACR response criteria measure changes from baseline in the number of tender and swollen joints, acute-phase response, a functional measure (e.g. HAQ score), visual analogue scale for pain, and global assessment of disease by patient and physician, also on a visual analogue scale. A 20%, 50% and 70% improvement in swollen and tender joint counts, and in the three of the remaining five parameters, respectively represent ACR20, ACR50 and ACR70 responses (Arnett et al. 1988). Therefore when the disease is rheumatoid arthritis, a preferred responder group of patients that provides for the reference values is a group that shows a significant change of ACR criteria and/or DAS. For example a DAS28≧1.2 after three months of treatment with a TNF-alpha blocking agent such as adalimumab (ADA) is indicative of a significant relief in the disease. Respectively an ACR50 after 12 weeks of treatment with a TNF-alpha blocking agent such as adalimumab (ADA) is indicative of a significant relief in the disease.

As intended herein “a lessened likelihood of responsiveness of said patient to a treatment with a TNF-alpha blocking agent with respect to standard responsiveness” means that the probability that a patient, e.g. with RA, which is homozygotous for the −238G, −308G, −857C haplotype will be responsive to treatment a TNF-alpha blocking agent is lower than that observed for a general population of patients with the same pathology, e.g. RA. As intended herein a “general population of patients” denotes a population of unselected patients, in particular as regards their TNF-alpha genotype. Preferably, the general population comprises enough patients so that the ratio of patients who respond to the treatment can be considered as statistically significant.

In particular, the probability that a patient, e.g. with RA, which is homozygtous for the −238G, −308G, −857C haplotype will be responsive to treatment a TNF-alpha blocking agent is lower than that observed for a population of patients with the same pathology who are not homozygous for the −238G, −308G, −857C haplotype.

For instance, as demonstrated in the following Example, the probability that RA patients homozygous for the −238G, −308G, −857C haplotype are responsive to treatment with a TNF-alpha blocking agent is of about 35%, whereas this probability is of about at least 40% in a general population of patients, or of about at least 50% among patients non homozygous for the −238G, −308G, −857C haplotype.

As intended herein patients who do not simultaneously carry, a guanine at position −238, a guanine at position −308 and a cytosine at position −857 of the TNF-alpha gene in both copies of said TNF-alpha gene of said patient, may particularly carry at least one of an adenine at position −238, an adenine at position −308, and a thymine at position −857 on at least one copy of said TNF-alpha gene of said patient.

### Method of the Invention

The method of the invention is based on the identification of a particular haplotype whose presence in a homozygous form allows distinguishing patients between responder and non-responder to a treatment with a TNF-alpha blocking agent.

Preferably, the haplotype of a patient is determined on a nucleic acid sample taken from said patient.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood or lymph, etc: DNA may be extracted using any methods known in the art, such as described in Sambrook et al., 1989.

The SNPs may be detected the nucleic acid sample, preferably after amplification. For instance, the isolated DNA may be subjected amplification by polymerase chain reaction (PCR), using oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular mutation. Otherwise, DNA may be amplified, after which a mutated site may be detected in the amplified sequence by hybridization with a suitable probe or by direct sequencing, or any other appropriate method known in the art.

Actually numerous strategies for genotype analysis are available. Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography. Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

The SNPs of the invention may be identified by using DNA chip technologies as those described in documents WO 2004/106546 and WO 2006/001627.

Nucleic acids having at least 10 nucleotides and exhibiting sequence complementarity or homology to the sequence of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, and enzymatic or other ligands (e.g. avidin/biotin). Probes typically comprise single-stranded nucleic acids of between 10 to 1000 nucleotides in length, for instance of between 10 and 800, more preferably of between 15 and 700, typically of between 20 and 500.

Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

According to another aspect of the invention, the mutations of interest are detected by contacting the nucleic sample of the patient with a nucleic acid probe, which is optionally labeled. Primers may also be useful to amplify or sequence the portion of the TNF-alpha gene (e.g. SEQ ID NO:1) containing the mutated positions of interest.

Such probes or primers are nucleic acids that are capable of specifically hybridizing with a portion of the TNF-alpha gene sequence (e.g. SEQ ID NO: 1) containing the mutated positions of interest. That means that they are sequences that hybridize with the portion mutated TNF-alpha nucleic acid sequence to which they relate under conditions of high stringency.

Oligonucleotide probes or primers may contain at least 10, 15, 20 or 30 nucleotides. Their length may be shorter than 400, 300, 200 or 100 nucleotides.

### TNF-Alpha Blocking Agents

In a particular embodiment TNF-alpha blocking agents include recombinant TNF-receptor based proteins (e.g. etanercept, a recombinant fusion protein consisting of two soluble TNF-alpha receptors joined by the Fc fragment of a human IgG1 molecule). A pegylated soluble TNF type 1 receptor can also be used as a TNF blocking agent. Additionally, thalidomide has been demonstrated to be a potent anti-TNF agent. TNF-alpha blocking agents thus further include phosphodiesterase 4 (IV) inhibitor thalidomide analogues and other phosphodiesterase IV inhibitors. In a particular embodiment the TNF-alpha blocking agent is a soluble form of a TNF-alpha receptor or an anti-TNF-alpha antibody, such as infliximab, adalimumab, or CDP571. In another particular embodiment, the TNF-alpha blocking agent is selected from the group constituted of etanercept, infliximab, and adalimumab. In a most preferred embodiment, the TNF-alpha blocking agent is adalimumab.

### TNF-Alpha-Related Disease

In particular embodiment the patient is affected with a TNF-alpha-related disease.

TNF-alpha-related disease may include an autoimmune disorder, an infectious disease, a transplant rejection or graft-versus-host disease, a malignancy, a pulmonary disorder, an intestinal disorder, a cardiac disorder, sepsis, a spondyloarthropathy, a metabolic disorder, an anemia, pain, a hepatic disorder, a skin disorder, a nail disorder, and a vasculitis. In one embodiment, the autoimmune disorder is selected from the group consisting of rheumatoid arthritis, rheumatoid spondylitis, osteoarthritis, gouty arthritis, allergy, multiple sclerosis, autoimmune diabetes, autoimmune uveitis, and nephrotic syndrome. In another embodiment, the TNF-alpha-related disease is selected from the group consisting of inflammatory bone disorders, bone resorption disease, alcoholic hepatitis, viral hepatitis, fulminant hepatitis, coagulation disturbances, burns, reperfusion injury, keloid formation, scar tissue formation, pyrexia, periodontal disease, obesity, and radiation toxicity. In still another embodiment, the TNF-alpha-related disease is selected from the group consisting of Behcet's disease, ankylosing spondylitis, asthma, chronic obstructive pulmonary disorder (COPD), idiopathic pulmonary fibrosis (IPF), restenosis, diabetes, anemia, pain, a Crohn's disease-related disorder, juvenile rheumatoid arthritis (JRA), a hepatitis C virus infection, psoriatic arthritis, and chronic plaque psoriasis.

In one embodiment of the invention, the TNF-alpha related disease is Crohn's disease. In another embodiment, the disease is ulcerative colitis. In still another embodiment, the disease is psoriasis. In still another embodiment, the disease is psoriasis in combination with psoriatic arthritis (PsA).

In the preferred embodiment, the TNF-alpha-related disease is rheumatoid arthritis.

The method of the invention is particularly useful to predict the response to a treatment by a TNF-alpha blocking agent in a patient with rheumatoid arthritis that is active.

Patients who are resistant to methotrexate (MTX), usually considered first-line therapy for the treatment of RA, are a further preferred group of patients for whom the method of the invention can be particularly useful.

More generally, patients who already receive a basic treatment for their TNF-alpha-related disease, e.g. with MTX, azathioprine or leflunomide, are particularly good candidates for the test method of the invention.

After being tested for responsiveness to a treatment with TNF-alpha blocking agent, the patients may be prescribed with a TNF-alpha blocking agent with or without the same basic treatment. In particular the combination adalimumab/MTX can be particularly effective in patients with RA and other TNF-alpha-related disease.

### Kits

The present invention further provides kits suitable for determining the haplotype of the invention.

The kits may include the following components:

(i) a probe, usually made of DNA, and that may be pre-labelled. Alternatively, the probe may be unlabelled and the ingredients for labelling may be included in the kit in separate containers; and

(ii) hybridization reagents: the kit may also contain other suitably packaged reagents and materials needed for the particular hybridization protocol, including solid-phase matrices, if applicable, and standards.

In another embodiment, the kits may include:

(i) sequence determination or amplification primers: sequencing primers may be pre-labelled or may contain an affinity purification or attachment moiety; and

(ii) sequence determination or amplification reagents: the kit may also contain other suitably packaged reagents and materials needed for the particular sequencing amplification protocol. In one preferred embodiment, the kit comprises a panel of sequencing or amplification primers, whose sequences correspond to sequences adjacent to at least one of the polymorphic positions, as well as a means for detecting the presence of each polymorphic sequence.

In a particular embodiment, it is provided a kit which comprises a pair of nucleotide primers specific for amplifying the TNF-alpha gene promoter comprising at least one of mutated that are identified herein, especially positions −238, −308 and −857 in the TNF gene.

### BRIEF DESCRIPTION OF THE FIGURES

FIG. 1 represents a flow chart of the exemplified pharmacogenetic study.

FIG. 2 represents ACR50 (50% improvement in symptoms according to the American College of Rheumatology criteria), 12 weeks after beginning of treatment of with adalimumab of RA patients having the −238G, −308G, and −857C haplotype (GGC) with respect to the TNF-alpha gene or another haplotype.

FIG. 3 shows the time course evolution (horizontal axis, weeks) of ACR50 response (vertical axis, % of ACR50 responder patients) according to treatment (with or without MTX) and GGC haplotype carrier status.

### EXAMPLE

### Methods

Patients: This pharmacogenetic study was ancillary from the ReAct (Research in Active Rheumatoid Arthritis) protocol performed at varied sites in Europe and Australia. In the parent ReAct study, 6610 patients were included to assess the safety and effectiveness of adalimumab (ADA), a fully human IgG1 anti-TNF monoclonal antibody. The objectives of the ReAct study were to evaluate efficacy and tolerance of ADA in combination with a variety of disease modifying anti-rheumatic drugs (DMARDs), including patients previously treated with etanercept or infliximab. Briefly, patients enrolled in the ReAct study were men and women years of age with active, adult-onset RA in accordance with the 1987 revised criteria of the American College of Rheumatology (ACR) (Arnett et al. 1988). Inclusion criteria required a disease duration of ≧3 months; a Disease Activity Score based on erythrocyte sedimentation rate and an evaluation of 28 joints (DAS28) (13) of indicating at least moderate disease activity; and treatment failure with at least 1 traditional DMARD. Previous therapy with biologic response modifiers including other TNF antagonists was allowed if the medication was discontinued months before enrolment.

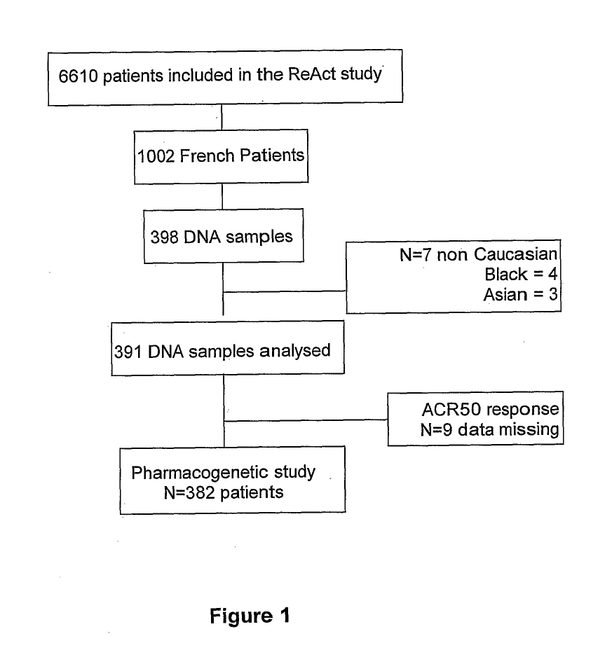

The pharmacogenetic study described herein included a large cohort of French patients. All the patients included provided written informed consent. The study was approved by the local ethic committee. In the main analysis, corresponding to the primary outcome variable (achievement of an ACR50 response after 12 weeks of treatment), seven patients included in the clinical trial were excluded from this pharmacogenetic study owing to their Asian or African descent (FIG. 1). Nine other patients were excluded from the statistical analysis owing to the lack of data on responses to treatment. Thus, a total of 382 patients from the original population were eligible for this study. Among these patients, ADA was associated with MTX (n=186) and administrated without MTX for the others (n=196).

Collection of clinical and biological data and Outcome measures: The clinical and biological collected data were those from the original ReAct protocol. At baseline, week 2, 6, 12, all the variables necessary to assess DAS 28 and ACR response were recorded as well as HAQ score. The primary outcome chosen for the genetic study was ACR50 response after 12 weeks of treatment. The other response data recorded at week 12 were ACR20, and ACR70 responses.

Genetic polymorphisms: The TNF gene polymorphisms analyzed were chosen according to a previous report which evidenced four main haplotypes constituted by TNF+488, −238 and −308 single-nucleotide polymorphisms (SNPs) in the Caucasian population. Nevertheless, as TNF+488 has been reported to be in strong linkage disequilibrium (LD) with TNF−857 (LD value, D′=0.92 in the Caucasian population (Simmonds et al. 2004)) and since TNF −857 was recently reported to influence clinical response to etanercept (Kang et al. 2005), we decided to genotype

TNF −857 instead of TNF+488. As expected, after haplotypic reconstruction, we also found four main haplotypes in our Caucasian population of RA patients. As specific HLA-DRB1 alleles have been previously reported to play an important role in RA susceptibility—the shared epitope hypothesis (SE) (Gregersen et al. 1987)—and severity, we also genotyped RA patients for HLA-DRB1 alleles by direct sequencing. The alleles considered to have the SE were HLA-DRB1 *0101, *0102, *0401, *0404, *0405, *0408, *0413, *1001, and *1402 (Gregersen et al. 1987). To analyze the SE contribution in response to ADA treatment, patients were classified as having 0, 1 or 2 copies of the SE or as being or not SE carriers. Extended haplotypes comprising HLA-DRB1 alleles and TNF SNPs were also reconstructed.

Genotyping methods: Patients were genotyped for HLA-DRB1 and 3 TNF-alpha gene polymorphisms (−238A/G, −308A/G and −857C(T). HLA-DRB1 alleles were determined by polymerase chain reaction (PCR) amplification and DNA sequencing using an ABI 3700 sequencer (PE Applied Biosystems, Foster City, Calif.).

TNF-alpha −857C/T was genotyped by allelic discriminating TaqMan PCR according to the procedure provided herein. Primers used were 5′ GGTCCTGGAGGCTCTTTCACT 3′ (SEQ ID NO: 2) and 5′ AGAATGTCCAGGGCTATGAAAGTC 3′ (SEQ ID NO: 3). Probes used herein were 5′ CCCTGTCTTCATTAAG (SEQ ID NO: 4) for the wild type and 5′ CCCTGTCTTCGTTAAG (SEQ ID NO: 5) for the mutant.

TNF −238A/G PCR gene polymorphisms was genotyped by mismatch polymerase chain reaction (PCR)-restriction full length polymorphism (RFLP) using the Mspl restriction enzyme. Primers used for PCR amplification were: forward 5′ATCTGGAGGAAGCGGTAGTG 3′ (SEQ ID NO: 6) and reverse 5′AGAAGACCCCCCTCGGAACC3′ (SEQ ID NO: 7). Reverse primer contained a purposeful mismatch sequence, so that when incorporated into the PCR products they create a Mspl with the G allele but not with the A allele.

TNF −308A/G was genotyped by allelic discriminating TaqMan PCR using the PreDeveloped TaqMan assay kit C_{—}7514879. Amplifications were performed using a 7900HT Applied Biosystems realtime thermal cycler (Applied Biosystems, Courtaboeuf, France).

Statistical analysis: All quantitative data are expressed as the mean+/−SD. All qualitative data are expressed as frequencies and percentages. Univariate regressions were performed to screen candidate covariates. The model with and without covariates were then compared using a χ² test. A multivariate regression model was then built including all candidate covariates selected in the previous analyse. The threshold for retaining a covariate in the model was 0.05.

For each gene, genotypes and haplotypes were tested for association with ACR50 response to ADA at Week 12. All genotyped SNPs were in Hardy-Weinberg equilibrium. Differences in genotype distribution for efficacy were tested using 3×2 crosstabs for each genotype, and using 2×2 crosstabs for each possible combination of homozygote and heterozygote genotypes, with the 2-sided chi-square test.

Within TNF gene, a measure of the LD between the different SNPs was estimated using Somers' D′. As there was a significant LD between all TNF SNPs and between TNF SNPs and HLA-DRB1 alleles, we also considered the haplotypes for TNF and extended haplotypes comprising HLA DRB1 alleles and TNF. We used the software PHASE (version 2.1) to perform haplotype reconstructions. This Bayesian algorithm provides the most likely pairs of haplotypes carried by each subjects (Stephens et al. 2001, Stephens et al. 2003). The average probability of PHASE certainty in haplotype inference was 99% for TNF haplotypes and 83% for SE-TNF extended haplotypes. Because the SE and 3 SNPs in the TNF locus were explored and because haplotype reconstructions were performed, a Bonferroni correction was applied for multiple comparisons. Both adjusted and unadjusted P values are presented. P values less than 0.05 were considered significant.

Results:

Description of the cohort: The baseline characteristics of patients are presented in Table 1.

TABLE 1
Baseline characteristics of 382 genotyped patients:

| | Baseline value |
|---|---|
| Characteristics | All patients (N = 382) |
| Sex, % female/% male | 78.3/21.7 |
| Age, mean +/− SD years | 53.8 +/− 11.3 |
| Disease duration, mean | 139.9 |
| weeks | |
| MDAS, mean +/− SD (range) | 5.9 +/− 1.0 |
| | (3.2-8.9) |
| No. of tender joints (1-28) +/− | 13.2 +/− 6.5 |
| SD | |
| No. of swollen joints (1-28) +/− | 9.8 +/− 5.0 |
| SD | |
| RF positivity, % | 70.9 |
| CRP, mean mg/liter (range) | 26.5 (3.5-167.0) |
| ESR, mean mm/hour (range) | 31.5 (2.0-98.0) |

MDAS = Modified Disease Activity Score based on erythrocyte sedimentation rate and an evaluation of 28 joints;
ESR = erythrocyte sedimentation rate;
RF = rheumatoid factor;
CRP = C-reactive protein

The profile of clinical response of the 382 patients included in this pharmacogenetic study was the same as that of entire ReAct population (6,610 patients). At 12 weeks, 41% of the patients (n=152) were ACR50 responders, 70% (n=267) were ACR20 responders and 15% (n=59) were ACR70 responders. MTX adjunction to ADA was significantly associated with a better ACR50 response both in univariate (P=0.005) and multivariate analyses (odds ratio: 1.76; 95% Cl: 1.14-2.74).

Genotype distributions were as follow: for TNF −238 G>A 94.4% GG, 5.3% AG, one patient had the rare AA genotype; for −308G>A 70% GG, 26.5% AG, 2.5% AA; for −857 C>T: 82.7% CC, 17% CT, one patient had the rare TT genotype. These distributions were consistent with those from public databases on the Caucasian population (http://www.ncbi.nlm.nih.gov/projects/SNP).

The distribution of the SE among patients was as follow: 0 copy 25.4%, 1 copy 47.6%, 2 copies 27%. Such distribution resulted in SE carriers 74.6% and non-SE carriers 25.4%.

Four main haplotypes were constructed with 3 SNPs in the TNF-alpha promoter, at position −238, −308 and −857 (e.g. the haplotype GGC consisted of −238G, −308G and −857C). These most frequent haplotypes (GGC, GAC, GGT and AGC) accounted for more than 99% of the total, with frequencies of 73, 15, 9 and 3% respectively. The rare AAC haplotype was found only in one patient.

Influence of the SE and individual TNF-alpha genotypes on ADA response: We found no correlation between ACR50 response to ADA at week 12 and the copy number of the SE or the SE carrier status (Table 2).

TABLE 2
Genotype frequencies of TNFA gene polymorphisms and HLA DRB1 SE
among ADA ACR50 responders and non responders at week 12.

| | | ACR50 response | | |
|---|---|---|---|---|
| | | | Non- | Uncorrected |
| Polymorphism | Genotypes | Responders | responders | p-value |
| TNFA- | CC | 114 (39%) | 178 (61%) | 0.12 |
| −857C/T | CT/TT | 31 (51%) | 30 (49%) | |
| N = 353 | | | | |
| TNFA | GG | 99 (38%) | 161 (62%) | 0.15 |
| −308G/A | GA/AA | 49 (46%) | 57 (54%) | |
| N = 366 | | | | |
| TNFA | GG | 134 (40%) | 202 (60%) | 0.12 |
| −238G/A | GA/AA | 12 (60%) | 8 (40%) | |
| N = 356 | | | | |
| HLA DRB1 | SE−/SE− | 28 (35%) | 53 (65%) | 0.31 |
| N = 319 | SE−/SE+ | 68 (45%) | 84 (55%) | |
| | SE+/SE+ | 34 (40%) | 52 (60%) | |
| | SE− | 28 (35%) | 53 (65%) | 0.19 |
| | SE+ | 102 (43%) | 136 (57%) | |

We found no correlation between ACR50 response to ADA at week 12 and any of the 3 TNF-alpha gene polymorphisms (−238A/G, −308A/G and −857C/T) genotypes neither (Table 2). Nevertheless, a trend toward an association between −238GG, −308GG, and −857CC genotypes and a poorer response to ADA was observed (Table 2).

Influence of the TNF-alpha haplotypes on ADA response: When the genetic effect of TNFA haplotypes on ADA response was analysed, we observed significant response differences according to the GGC haplotype carrier status (Table 3).

TABLE 3
Haplotype combinations frequencies among ADA ACR50
responders and non responders at week 12.

| | ACR50 RESPONSE | | |
|---|---|---|---|
| Haplotypes | | | Uncorrected p- |
| combination* | Responders | Non-responders | value |
| GGC/AGC | 10 (71%) | 4 (29%) | 0.0041 |
| N = 14 | | | |
| GGC/GGT | 24 (53%) | 21 (47%) | |
| N = 45 | | | |
| GGC/GAC | 36 (47%) | 41 (53%) | |
| N = 77 | | | |
| GGC/GGC | 62 (34%) | 122 (66%) | |
| N = 184 | | | |

*Haplotypes are written in the order −238, −308 and −857 SNPs of TNF-alpha.

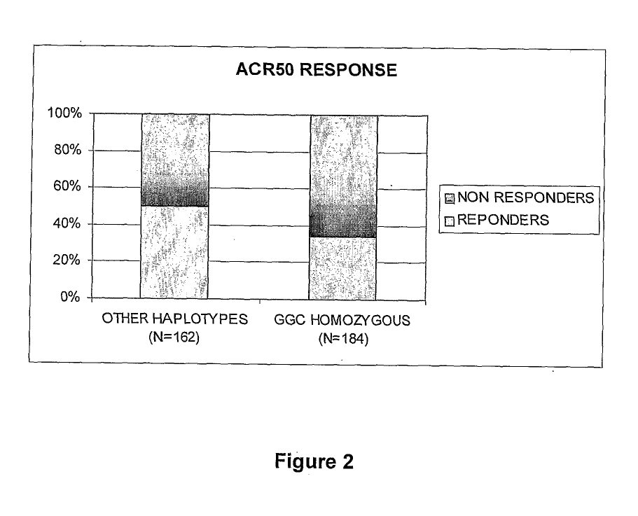

In the first analyse, we discarded rare haplotype combinations (N=27), represented less than 10 times: AGC/AAC (N=1), GAC/AGC (N=2), GAC/GAC (N=9), GGT/AGC (N=3), GGT/GAC (N=9), GGT/GGT (N=3). In the remaining four main haplotype combinations, homozygous individuals for GGC haplotypes (N=184) had a significant lowest ACR50 response rate (34%) compared with each of the three other more frequent combined haplotypes: GGC/GAC 47% (N=77), GGC/GGT 53% (N=45), and GGC/AGC 71% (N=14) (P=0.0041, Pc=0.02) (Table 3). Such observation was highly suggestive of a recessive effect of the GGC haplotype on response to treatment. In fact, the response rates between GGC haplotype homozygous carriers was 33% and significantly lower than the 50% response rate observed for all the other haplotype combinations (P=0.003, Pc=0.015) (FIG. 2). ACR20 and ACR70 response at week 12 were secondary endpoint criteria not significantly influenced by GCC homozygosity, even if a similar trend was observed in both groups: 69% response in GGC/GGC group versus 76% among the other haplotypes carriers (P=0.14) and 15% response in GGC/GGC group versus 19% among the other haplotypes carriers (P=0.3), respectively. The lack of significant difference in these groups is probably explained by a loss of power due to an unbalanced distribution of patients between responders and non responders. Conversely, ACR50 response at week 12 provides the best statistical power to demonstrate an effect with a distribution of responders and non responders neighbouring 50% of the whole population.

We next search for baseline characteristics differences between GGC homozygous patients and the others haplotypes carriers that may interfered with our results. There were no significant differences between both groups, especially on DAS28 RA disease activity criteria (Table 4).

TABLE 4
Comparisons between patients homozygous for GGC haplotype
and the other patients for the main RA characteristics.

| | GCC homozygous | | |
|---|---|---|---|
| Variable | (N = 184) | Other (N = 162) | P-value |
| Disease duration, | 141 | 139 | 0.93 |
| mean weeks | | | |
| RF positivity, % | 72 | 71 | 0.90 |
| Age, mean | 54.6 | 53.4 | 0.27 |
| MDAS, mean | 5.88 | 5.86 | 0.88 |
| No. of tender joints | 13.2 | 13.3 | 0.91 |
| (1-28) | | | |
| No. of swollen | 9.6 | 10.30 | 0.10 |
| joints (1-28) | | | |
| ESR, mean | 32.12 | 30.57 | 0.53 |
| mm/hour (range) | | | |
| CRP, mean mg/liter | 25.9 | 28.27 | 0.34 |
| (range) | | | |
| SE, % (0, 1 and 2 | 21/43/36 | 32/50/18 | 0.0012 |
| copies) | | | |
| MTX+, % | 43 | 51 | 0.17 |

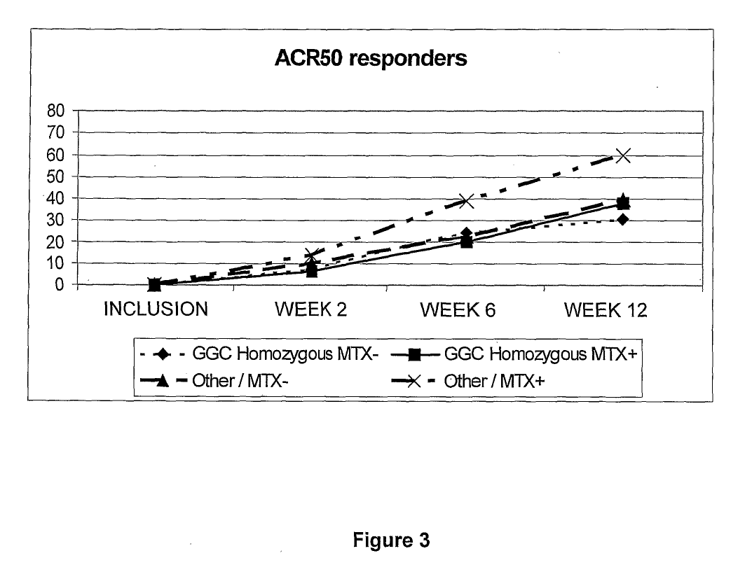

As about half of the studied population was taking MTX treatment, we further analyzed the GGC homozygosity effect on each treatment subgroup (with or without MTX). Surprisingly, the poorer response associated with GGC homozygosity was present mainly in the group of patients taking MTX: in this group, ACR50 response rate was 38% among GGC haplotype homozygous carriers versus 60% among the other haplotype carriers (P=0.0095; Pc=0.047). In the group without MIX, ACR50 response rate among GGC haplotype homozygous carriers (30%) was lower than among the other haplotype carriers (40%), but the difference between both groups was not significant (P=0.25). The time course evolution of ACR50 response in each treatment groups was very interesting (FIG. 3). In fact, among MTX treated patients, response difference between GGC haplotype homozygous carriers and the other haplotype carriers was identifiable as soon as week 2 and still increased until week 12. In the group without MTX, the response difference between GGC/GGC carriers and the other patients appeared later, around week 10, and with a lower magnitude, suggesting that the trend in favour of a significant difference between response rates at week 12 in the group without MTX could become a true significant difference with a longer follow-up unfortunately not available in the ReAct protocol.

Influence of the SE on the negative effect of GGC homozygosity on ADA response: TNFA locus is located in the close vicinity of HLA DRB1, so we wanted to analyze to what extent GGC haplotype was in LD with some alleles belonging to the SE. The distribution of copy number of the SE was significantly different between GGC homozygous patients and the other patients (P=0.0012) and thus suggestive of a LD between GGC haplotype and alleles from the SE (Table 4).

Nevertheless, such distribution was not based on extended haplotype reconstruction and could reflect cis- as well as trans-association of the SE alleles with GGC haplotypes. After extended haplotype reconstructions, 60% of the GGC haplotypes were associated with an allele of the SE compared with only 28% of the non-GGC haplotypes (P=2.10⁻¹²). LD between GGC haplotype and alleles of the SE was therefore confirmed. Extended haplotype reconstructions led to 82 SE-TNF haplotype combinations. A careful examination of these extended haplotypes showed that GGC haplotype was mainly associated with HLA DRB1*0101 and *0401 alleles of the SE, corresponding to the most frequent HLA DRB1 alleles among Caucasians (Table 5).

TABLE 5
Extended HLA DRB1-TNF haplotype reconstructions. Haplotypes
carried by as least 10 patients have been detailed.

| Extended | Haplotype | ACR50 response at |
|---|---|---|
| HLA DRB1-TNF | frequency, % | week 12, % (n) |
| haplotypes | (n) | (1 copy carriers) |
| 0101-GGC | 13% (84) | 41% (80) |
| 0401-GGC | 16% (98) | 42% (88 |
| 0404-GGC | 6.5% (41) | 38% (40) |
| 0405-GGC | 4% (24) | 30% (23) |
| 0701-GGC | 5% (30) | 24% (29) |
| 1001-GGC | 2% (12) | 42% (12) |
| 1101-GGC | 4% (24) | 39% (23) |
| 1302-GGC | 2% (11) | 36% (11) |
| 1501-GGC | 4.5% (28) | 37% (27) |
| Other | 50.4% (317) | — — |

Most of these extended haplotypes were associated with an around 40% ACR50 response rate (range 36-42), except for the 0701-GGC haplotype (24% response) and the 0405-GGC haplotype (30% response).

At this stage of the study, it was important to analyse if the poorer response observed among GGC homozygous haplotype carriers could be due to the LD with alleles of the SE. The rate of ACR50 responders at week 12 among the carriers of 2 copies of the SE was 40% and was non-significantly different from the response rate among carriers of 0 or 1 copy of the SE (41%). Thus, the lack of association between the SE, even carried on both chromosomes, and ACR50 poor response to ADA at week 12, pleads against such an hypothesis. Moreover, GGC homozygous haplotype carriers responded equally to ADA when patients carried (37%) or not (27%) the SE (P=0.43).

Conclusions:

This pharmacogenetic study is remarkable because of the size of the population as well as the quality of the clinical data recorded within the ReAct study. It provides robust data indicating that a single TNF-alpha locus haplotype (−238G/−308G/−857C) when carried on both chromosomes is associated with a poorer response to ADA in RA patients according to a recessive mode which is not attributable to the specific SE alleles.

### REFERENCES

Arnett F C, Edworthy S M, Bloch D A, McShane D J, Fries J F, Cooper N S, et al. The American Rheumatism Association 1987 revised criteria for the classification of rheumatoid arthritis. Arthritis Rheum 1988; 31(3):315-24.
Criswell L A, Lum R F, Turner K N, Woehl B, Zhu Y, Wang J, et al. The influence of genetic variation in the HLA-DRB1 and LTA-TNF regions on the response to treatment of early rheumatoid arthritis with methotrexate or etanercept. Arthritis Rheum 2004; 50(9):2750-6.
Cuchacovich M, Ferreira L, Aliste M, Soto L, Cuenca J, Cruzat A, et al. Tumour necrosis factor-alpha (TNF-alpha) levels and influence of −308 TNF-alpha promoter polymorphism on the responsiveness to infliximab in patients with rheumatoid arthritis. Scand J Rheumatol 2004; 33(4):228-32.
Fonseca J E, Carvalho T, Cruz M, Nero P, Sobral M, Mourao A F, et al. Polymorphism at position −308 of the tumour necrosis factor alpha gene and rheumatoid arthritis pharmacogenetics. Ann Rheum Dis 2005; 64(5):793-4.
Gregersen P K, Silver J, Winchester R J. The shared epitope hypothesis. An approach to understanding the molecular genetics of susceptibility to rheumatoid arthritis. Arthritis Rheum 1987; 30(11):1205-13.
Kang C P, Lee K W, Yoo D H, Kang C, Bae S C. The influence of a polymorphism at position −857 of the tumour necrosis factor alpha gene on clinical response to etanercept therapy in rheumatoid arthritis. Rheumatology (Oxford) 2005; 44(4):547-52.
Lee Y H, Rho Y H, Choi S J, Ji J D, Song G G. Association of TNF-alpha −308 G/A polymorphism with responsiveness to TNF-alpha-blockers in rheumatoid arthritis: a meta-analysis. Rheumatol Int 2006.
Marotte H, Pallot-Prades B, Grange L, Tebib J, Gaudin P, Alexandre C, Blond J L, Cazalis M A, Mougin B, Miossec P. The shared epitope is a marker of severity associated with selection for, but not with response to, infliximab in a large rheumatoid arthritis population. Ann Rheum Dis. 2006, 65:342-7
Martinez A, Salido M, Bonilla G, Pascual-Salcedo D, Fernandez-Arquero M, de Miguel S, et al. Association of the major histocompatibility complex with response to infliximab therapy in rheumatoid arthritis patients. Arthritis Rheum 2004; 50(4):1077-82.
Mascheretti S, Hampe J, Kuhbacher T, Herfarth H, Krawczak M, Folsch U R, et al. Pharmacogenetic investigation of the TNF/TNF-receptor system in patients with chronic active Crohn's disease treated with infliximab. Pharmacogenomics J 2002; 2(2):127-36.
Mugnier B, Balandraud N, Darque A, Roudier C, Roudier J, Reviron D. Polymorphism at position −308 of the tumor necrosis factor alpha gene influences outcome of infliximab therapy in rheumatoid arthritis. Arthritis Rheum 2003; 48(7):1849-52.
Mugnier B, Roudier J. Tumor necrosis factor alpha haplotypes versus tumor necrosis factor alpha −308 G/A polymorphism in the prediction of infliximab treatment efficacy in rheumatoid arthritis. Arthritis Rheum 2004; 50(12):4075-6; author reply 4076-7.
Padyukov L, Lampa J, Heimburger M, Ernestam S, Cederholm T, Lundkvist I, et al. Genetic markers for the efficacy of tumour necrosis factor blocking therapy in rheumatoid arthritis. Ann Rheum Dis 2003; 62(6):526-9.
Prevoo M L, van't Hof M A, Kuper E H, van Leeuwen M A, van de Putte L B, van Riel P L. Modified disease activity scores that include twenty-eight-joint counts. Development and validation in a prospective longitudinal study of patients with rheumatoid arthritis. Arthritis Rheum 1995, 38(1):44-8.
Simmonds M J, Heward J M, Howson J M, Foxall H, Nithiyananthan R, Franklyn J A, et al. A systematic approach to the assessment of known TNF-alpha polymorphisms in Graves' disease. Genes Immun 2004; 5(4):267-73.
Stephens J C, Schneider J A, Tanguay D A, Choi J, Acharya T, Stanley S E, et al. Haplotype variation and linkage disequilibrium in 313 human genes. Science 2001; 293(5529):489-93.
Stephens M, Donnelly P. A comparison of bayesian methods for haplotype reconstruction from population genotype data. Am J Hum Genet 2003; 73(5):1162-9.

1. A method for predicting the responsiveness of a patient to a treatment with a TNF-alpha blocking agent, said method comprising determining the presence or absence of a guanine at position −238, a guanine at position −308, and a cytosine at position −857 of the TNF-alpha gene of said patient, wherein the simultaneous presence of a guanine at position −238, a guanine at position −308, and a cytosine at position −857 of the TNF-alpha gene in both copies of said TNF-alpha gene of said patient is indicative of a lessened likelihood of responsiveness of said patient to a treatment with a TNF-alpha blocking agent with respect to standard responsiveness.

2. The method of claim 1, wherein the patient suffers from rheumatoid arthritis.

3. The method of claim 1, wherein the patient suffers from active rheumatoid arthritis.

4. The method of claim 1, wherein the TNF-alpha blocking agent is an anti-TNF-alpha antibody or a soluble form of a TNF-alpha receptor.

5. The method of claim 1, wherein the TNF-alpha blocking agent is selected from the group constituted of etanercept, infliximab, and adalimumab.

6. The method of claim 1, wherein the TNF-alpha blocking agent is adalimumab.

7. A method for the treatment of a TNF-alpha-related disease in an individual comprising administering the individual with a therapeutically effective quantity of a TNF-alpha blocking agent, wherein said individual does not simultaneously carry a guanine at position −238, a guanine at position −308, and a cytosine at position −857 of the TNF-alpha gene in both copies of said TNF-alpha gene.

8. The method of claim 7, wherein the TNF-alpha blocking agent is an anti-TNF-alpha antibody or a soluble form of a TNF-alpha receptor.

9. The method of claim 7, wherein the TNF-alpha blocking agent is selected from the group constituted of etanercept, infliximab, and adalimumab

10. The method of claim 7, wherein the TNF-alpha blocking agent is adalimumab.

11. The method of claim 7, wherein the TNF-alpha-related disease is rheumatoid arthritis.


SEQUENCE LISTING (5 sequences)

SEQ ID NO: 1 - DNA, 3908 bp, Homo sapiens
  misc_feature (33)..(33): c or t
tatggaagtcgagtatggggacccccccttaangaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatgnggacggggttcagcctccagggtcctacacacaaatcagtcagtggcccagaagacccccctcggaatcngagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagaggtgagtgcctggccagccttcatccactctcccacccaaggggaaatgagagacgcaagagagggagagagatgggatgggtgaaagatgtgcgctgatagggagggatgagagagaaaaaaacatggagaaagacggggatgcagaaagagatgtggcaagagatggggaagagagagagagaaagatggagagacaggatgtctggcacatggaaggtgctcactaagtgtgtatggagtgaatgaatgaatgaatgaatgaacaagcagatatataaataagatatggagacagatgtggggtgtgagaagagagatgggggaagaaacaagtgatatgaataaagatggtgagacagaaagagcgggaaatatgacagctaaggagagagatgggggagataaggagagaagaagatagggtgtctggcacacagaagacactcagggaaagagctgttgaatgctggaaggtgaatacacagatgaatggagagagaaaaccagacacctcagggctaagagcgcaggccagacaggcagccagctgttcctcctttaagggtgactccctcgatgttaaccattctccttctccccaacagttccccagggacctctctctaatcagccctctggcccaggcagtcagtaagtgtctccaaacctctttcctaattctgggtttgggtttgggggtagggttagtaccggtatggaagcagtgggggaaatttaaagttttggtcttgggggaggatggatggaggtgaaagtaggggggtattttctaggaagtttaagggtctcagctttttcttttctctctcctcttcaggatcatcttctcgaaccccgagtgacaagcctgtagcccatgttgtaggtaagagctctgaggatgtgtcttggaacttggagggctaggatttggggattgaagcccggctgatggtaggcagaacttggagacaatgtgagaaggactcgctgagctcaagggaagggtggaggaacagcacaggccttagtgggatactcagaacgtcatggccaggtgggatgtgggatgacagacagagaggacaggaaccggatgtggggtgggcagagctcgagggccaggatgtggagagtgaaccgacatggccacactgactctcctctccctctctccctccctccagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaacatggtctccttcttggaattaattctgcatctgcctcttcttgtgggtgggaagaagctccctaagtcctctctccacaggctttaagatccctcggacccagtcccatccttagactcctagggccctggagaccctacataaacaaagcccaacagaatattccccatcccccaggaaacaagagcctgaacctaattacctctccctcagggcatgggaatttccaactctgggaattc

SEQ ID NO: 2 - DNA, 21 bp, Artificial sequence
  PCR primer
ggtcctggaggctctttcact

SEQ ID NO: 3 - DNA, 24 bp, Artificial sequence
  PCR primer
agaatgtccagggctatgaaagtc

SEQ ID NO: 4 - DNA, 16 bp, Artificial sequence
  Detection probe
ccctgtcttcattaag

SEQ ID NO: 5 - DNA, 16 bp, Artificial sequence
  Detection probe
ccctgtcttcgttaag